(19) United States
(12) Patent Application Publication
(10) Pub. No.: US 2010/0016862 A1
(43) Pub. Date: Jan. 21, 2010

(54) METHOD OF PROVIDING EMBOLIC PROTECTION AND SHOCKWAVE ANGIOPLASTY THERAPY TO A VESSEL

(76) Inventors: Daniel Hawkins, Newcastle, WA (US); John M. Adams, Snohomish, WA (US)

Correspondence Address: GRAYBEAL JACKSON LLP, 155 - 108TH AVENUE NE, SUITE 350, BELLEVUE, WA, 98004-5973, US

(21) Appl. No.: 12/501,619

(22) Filed: Jul. 13, 2009

Related U.S. Application Data
(60) Provisional application No. 61/081,292, filed on Jul. 16, 2008.

(51) Int. Cl. A61B 17/221

(52) U.S. Cl. 606/128

(57) ABSTRACT

A system comprises a guide wire, an embolic protection basket carried on the guide wire, and a catheter carried on the guide wire adjacent the embolic protection basket. The catheter includes an elongated carrier and a balloon about the carrier in sealed relation thereto. The balloon is arranged to receive a fluid therein that inflates the balloon, and an arc generator including at least one electrode within the balloon forms a mechanical shock wave within the balloon.

### PRIORITY CLAIM

The present application claims the benefit of copending U.S. Provisional Patent Application Ser. No. 60/081,292 filed Jul. 16, 2008, which application is incorporated herein by reference in its entirety.

### BACKGROUND OF THE INVENTION

The present invention relates to a treatment system for percutaneous coronary angioplasty or peripheral angioplasty in which a dilation catheter is used to cross a lesion in order to dilate the lesion and restore normal blood flow in the artery. It is particularly useful when the lesion is a calcified lesion in the wall of the artery. Calcified lesions require high pressures (sometimes as high as 10-15 or even 30 atmospheres) to break the calcified plaque and push it back into the vessel wall. With such pressures comes trauma to the vessel wall which can contribute to vessel rebound, dissection, thrombus formation, and a high level of restenosis. Non-concentric calcified lesions can result in undue stress to the free wall of the vessel when exposed to high pressures. An angioplasty balloon when inflated to high pressures can have a specific maximum diameter to which it will expand but the opening in the vessel under a concentric lesion will typically be much smaller. As the pressure is increased to open the passage way for blood the balloon will be confined to the size of the open in the calcified lesion (before it is broken open). As the pressure builds a tremendous amount of energy is stored in the balloon until the calcified lesion breaks or cracks. That energy is then released and results in the rapid expansion of the balloon to its maximum dimension and may stress and injure the vessel walls.

During angioplasty therapy, plaque debris can be produced. If such debris were permitted to freely translate within the blood stream, it could potentially coagulate down stream and result in another vein or artery reduction. Hence, it is desirable to preclude such debris from being freely carried within the blood stream during the angioplasty procedure. The present invention addresses this and other issues.

### SUMMARY OF THE INVENTION

In one embodiment, the invention provides a system comprising a guide wire, an embolic protection basket carried on the guide wire, and a catheter carried on the guide wire adjacent the embolic protection basket. The catheter includes an elongated carrier and a balloon about the carrier in sealed relation thereto. The balloon is arranged to receive a fluid therein that inflates the balloon, and agenerator within the balloon forms a mechanical shock wave within the balloon.

The generator may be an arc generator and include at least one electrode that forms an electrical arc.

The embolic protection basket may be downstream from the catheter. The system may further include a power source that provides electrical energy to the arc generator. The embolic protection basket may be a collapsible structure.

The system may further comprise a push tube that pushes the embolic protection basket into position along the guide wire. The embolic protection basket may be a collapsible structure and the system may further comprise an over tube that maintains the embolic protection basket in a collapsed condition.

The system may further comprise a push tube that pushes the embolic protection basket into position along the guide wire. The embolic protection basket may be a collapsible structure and the system may further comprise an over tube that maintains the embolic protection basket in a collapsed condition as the push tube pushes the embolic protection basket into position along the guide wire.

The guide wire may include a stop that positions the embolic protection basket on the guide wire. The catheter may include a lumen for being received on the guide wire.

The invention further provides a method comprising providing a guide wire, inserting the guide wire into a vessel of interest of a patient, providing an embolic protection basket, advancing the embolic protection basket along the guide wire within the vessel, and providing a catheter including an elongated carrier, a balloon about the carrier in sealed relation thereto, the balloon being arranged to receive a fluid therein that inflates the balloon, and agenerator within the balloon that forms a mechanical shock wave within the balloon. The method further includes the steps of guiding the catheter into the vessel of the patient to a position adjacent to and up stream from the embolic protection basket, admitting fluid into the balloon, and causing the generator to form a series of mechanical shocks within the balloon.

The generator may be an arc generator including at least one electrode that forms electrical arcs to form the shockwave. The causing step may include the step of applying high voltage pulses to the generator.

The embolic protection basket may be a collapsible structure and the step of advancing the embolic protection basket along the guide wire within the vessel may be performed with the embolic protection basket maintained in a collapsed state. The embolic protection basket may be maintained in a collapsed state by enclosing the embolic protection basket within an over tube. The method may include the further step of releasing the embolic protection basket from the collapsed state by separating the embolic protection basket from the over tube prior to applying high voltage pulses to the arc generator to form a series of mechanical shocks within the balloon.

### BRIEF DESCRIPTION OF THE DRAWINGS

The various embodiments of the invention, together with representative features and advantages thereof, may best be understood by making reference to the following description taken in conjunction with the accompanying drawings, in the several figures of which like reference numerals identify identical elements, and wherein:

FIG. 1 is a view of the therapeutic end of a typical prior art over-the-wire angioplasty balloon catheter;

FIG. 2 is a side view of a dilating angioplasty balloon catheter with two electrodes within the balloon attached to a source of high voltage pulses according to one embodiment of the invention;

FIG. 3 is a schematic of a high voltage pulse generator;

FIG. 3A shows voltage pulses that may be obtained with the generator of FIG. 3;

FIG. 4 is a side view of the catheter of FIG. 2 showing an arc between the electrodes and simulations of the shock wave flow;

FIG. 5 is a side view of a dilating catheter with insulated electrodes within the balloon and displaced along the length of the balloon according to another embodiment of the invention;

FIG. 6 is a side view of a dilating catheter with insulated electrodes within the balloon displaced with a single pole in the balloon and a second being the ionic fluid inside the balloon according to a further embodiment of the invention;

FIG. 7 is a side view of a dilating catheter with insulated electrodes within the balloon and studs to reach the calcification according to a still further embodiment of the invention;

FIG. 8 is a side view of a dilating catheter with insulated electrodes within the balloon with raised ribs on the balloon according to still another embodiment of the invention;

FIG. 8A is a front view of the catheter of FIG. 8;

FIG. 9 is a side view of a dilating catheter with insulated electrodes within the balloon and a sensor to detect reflected signals according to a further embodiment of the invention;

FIG. 10 is a pressure volume curve of a prior art balloon breaking a calcified lesion;

FIG. 10A is a sectional view of a balloon expanding freely within a vessel;

FIG. 10B is a sectional view of a balloon constrained to the point of breaking in a vessel;

FIG. 10C is a sectional view of a balloon after breaking within the vessel;

FIG. 11 is a pressure volume curve showing the various stages in the breaking of a calcified lesion with shock waves according to an embodiment of the invention;

FIG. 11A is a sectional view showing a compliant balloon within a vessel;

FIG. 11B is a sectional view showing pulverized calcification on a vessel wall;

FIG. 12 illustrates shock waves delivered through the balloon wall and endothelium to a calcified lesion;

FIG. 13 shows calcified plaque pulverized and smooth a endothelium restored by the expanded balloon after pulverization;

FIG. 14 is a schematic of a circuit that uses a surface EKG to synchronize the shock wave to the “R” wave for treating vessels near the heart;

FIG. 15 is a side view, partly cut away, of a dilating catheter with a parabolic reflector acting as one electrode and provides a focused shock wave inside a fluid filled compliant balloon;

FIG. 16 is a side view, partly in section, of an embolic protection basket being guided into position in accordance with an embodiment of the invention;

FIG. 17 is a side view, partly in section, of the embolic protection basket after reaching its deployment position in accordance with an embodiment of the invention;

FIG. 18 is a side view, partly in section, of the embolic protection basket after being deployed in a vein or artery to be treated in accordance with an embodiment of the invention;

FIG. 19 is a side view, partly in section, of the embolic protection basket combined with a shockwave angioplasty device as angioplasty therapy is applied to the vein or artery in accordance with an embodiment of the invention; and

FIG. 20 is a side view, partly in section, of the embolic protection basket combined and shockwave angioplasty device being removed from the vein or artery after angioplasty therapy in accordance with an embodiment of the invention.

### DETAILED DESCRIPTION OF THE EMBODIMENTS

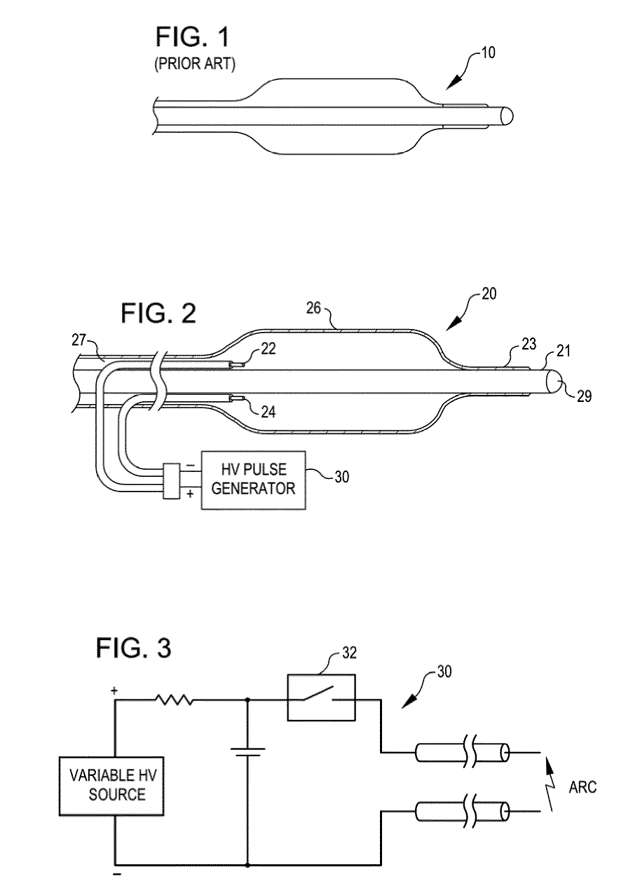

FIG. 1 is a view of the therapeutic end of a typical prior art over-the-wire angioplasty balloon catheter 10. Such catheters are usually non-complaint with a fixed maximum dimension when expanded with a fluid such as saline.

FIG. 2 is a view of a dilating angioplasty balloon catheter 20 according to an embodiment of the invention. The catheter 20 includes an elongated carrier, such as a hollow sheath 21, and a dilating balloon 26 formed about the sheath 21 in sealed relation thereto at a seal 23. The balloon 26 forms an annular channel 27 about the sheath 21 through which fluid, such as saline, may be admitted into the balloon to inflate the balloon. The channel 27 further permits the balloon 26 to be provided with two electrodes 22 and 24 within the fluid filled balloon 26. The electrodes 22 and 24 are attached to a source of high voltage pulses 30. The electrodes 22 and 24 are formed of metal, such as stainless steel, and are placed a controlled distance apart to allow a reproducible arc for a given voltage and current. The electrical arcs between electrodes 22 and 24 in the fluid are used to generate shock waves in the fluid. The variable high voltage pulse generator 30 is used to deliver a stream of pulses to the electrodes 22 and 24 to create a stream of shock waves within the balloon 26 and within the artery being treated (not shown). The magnitude of the shock waves can be controlled by controlling the magnitude of the pulsed voltage, the current, the duration and repetition rate. The insulating nature of the balloon 26 protects the patient from electrical shocks.

The balloon 26 may be filled with water or saline in order to gently fix the balloon in the walls of the artery in the direct proximity with the calcified lesion. The fluid may also contain an x-ray contrast to permit fluoroscopic viewing of the catheter during use. The carrier 21 includes a lumen 29 through which a guidewire (not shown) may be inserted to guide the catheter into position. Once positioned the physician or operator can start with low energy shock waves and increase the energy as needed to crack the calcified plaque. Such shockwaves will be conducted through the fluid, through the balloon, through the blood and vessel wall to the calcified lesion where the energy will break the hardened plaque without the application of excessive pressure by the balloon on the walls of the artery.

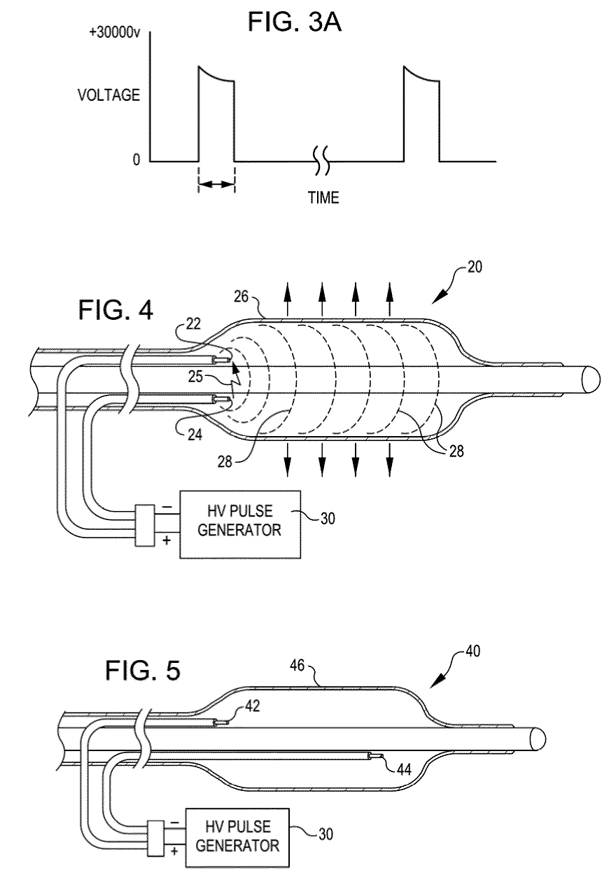

FIG. 3 is a schematic of the high voltage pulse generator 30. FIG. 3A shows a resulting waveform. The voltage needed will depend on the gap between the electrodes and generally 100 to 3000 volts. The high voltage switch 32 can be set to control the duration of the pulse. The pulse duration will depend on the surface area of the electrodes 22 and 24 and needs to be sufficient to generate a gas bubble at the surface of the electrode causing a plasma arc of electric current to jump the bubble and create a rapidly expanding and collapsing bubble, which creates the mechanical shock wave in the balloon. Such shock waves can be as short as a few microseconds.

FIG. 4 is a cross sectional view of the shockwave catheter 20 showing an arc 25 between the electrodes 22 and 24 and simulations of the shock wave flow 28. The shock wave 28 will radiate out from the electrodes 22 and 24 in all directions and will travel through the balloon 26 to the vessel where it will break the calcified lesion into smaller pieces.

FIG. 5 shows another dilating catheter 40. It has insulated electrodes 42 and 44 within the balloon 46 displaced along the length of the balloon 46.

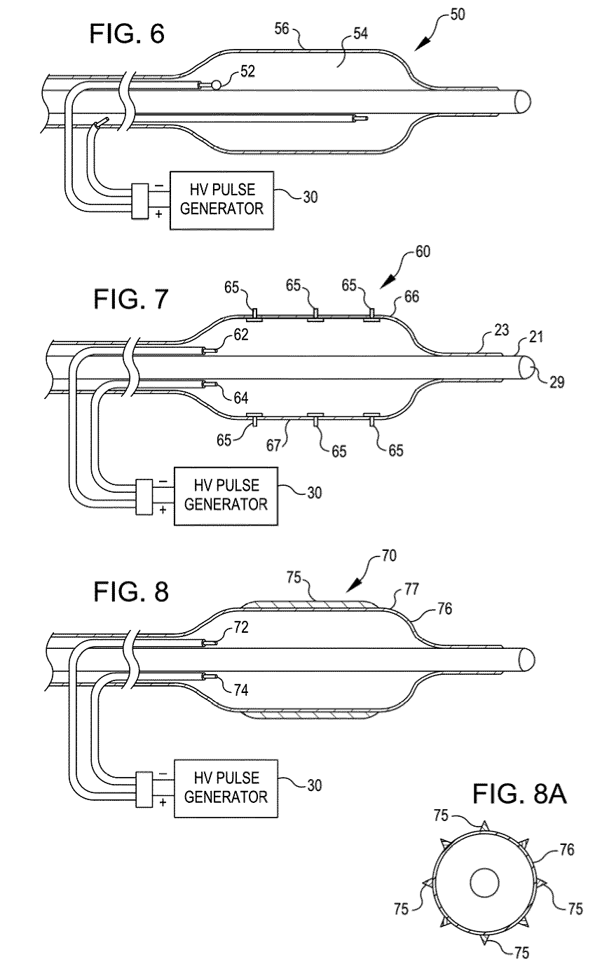

FIG. 6 shows a dilating catheter 50 with an insulated electrode 52 within the balloon 56. The electrode is a single electrode pole in the balloon, a second pole being the ionic fluid 54 inside the balloon. This unipolar configuration uses the ionic fluid as the other electrical pole and permits a smaller balloon and catheter design for low profile balloons. The ionic fluid is connected electrically to the HV pulse generator 30.

FIG. 7 is another dilating 60 catheter with electrodes 62 and 64 within the balloon 66 and studs 65 to reach the calcification. The studs 65 form mechanical stress risers on the balloon surface 67 and are designed to mechanically conduct the shock wave through the intimal layer of tissue of the vessel and deliver it directly to the calcified lesion.

FIG. 8 is another dilating catheter 70 with electrodes 72 and 74 within the balloon 76 and with raised ribs 75 on the surface 77 of the balloon 76. The raised ribs 75 (best seen in FIG. 8A) form stress risers that will focus the shockwave energy to linear regions of the calcified plaque.

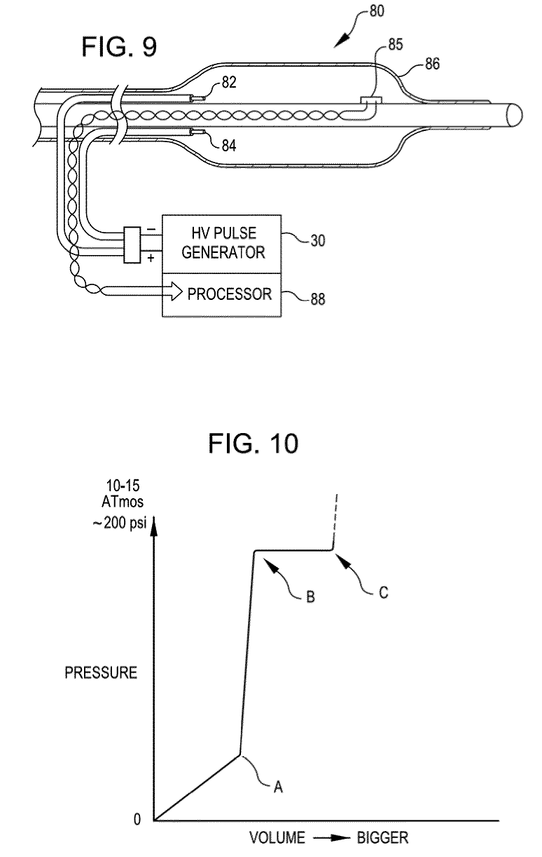

FIG. 9 is a further dilating catheter 80 with electrodes 82 and 84 within the balloon 86. The catheter 80 further includes a sensor 85 to detect reflected signals. Reflected signals from the calcified plaque can be processed by a processor 88 to determine quality of the calcification and quality of pulverization of the lesion.

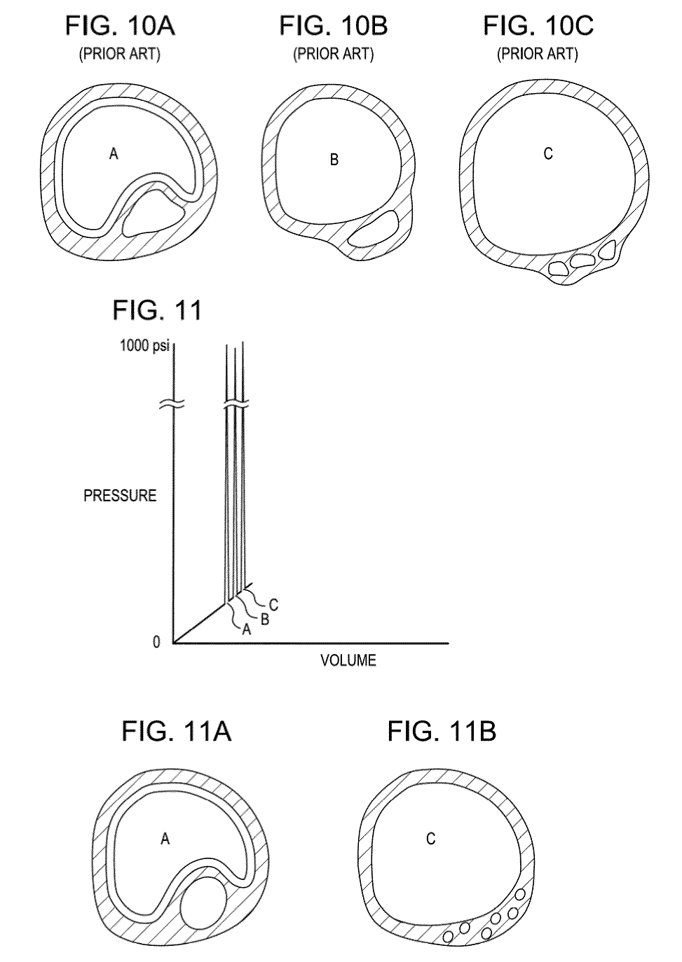

FIG. 10 is a pressure volume curve of a prior art balloon breaking a calcified lesion. FIG. 10B shows the build up of energy within the balloon (region A to B) and FIG. 10C shows the release of the energy (region B to C) when the calcification breaks. At region C the artery is expanded to the maximum dimension of the balloon. Such a dimension can lead to injury to the vessel walls. FIG. 10A shows the initial inflation of the balloon.

FIG. 11 is a pressure volume curve showing the various stages in the breaking of a calcified lesion with shock waves according to the embodiment. The balloon is expanded with a saline fluid and can be expanded to fit snugly to the vessel wall (Region A) (FIG. 11A) but this is not a requirement. As the High Voltage pulses generate shock waves (Region B and C) extremely high pressures, extremely short in duration will chip away the calcified lesion slowly and controllably expanding the opening in the vessel to allow blood to flow unobstructed (FIG. 11B).

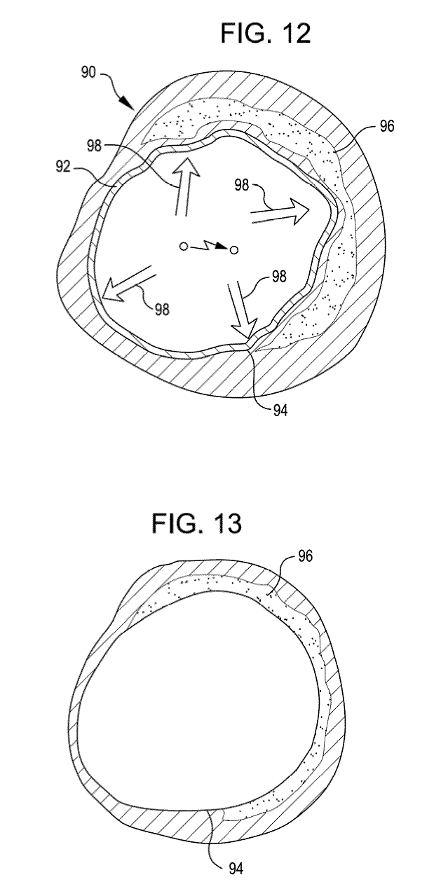

FIG. 12 shows, in a cutaway view, shock waves 98 delivered in all directions through the wall 92 of a saline filled balloon 90 and intima 94 to a calcified lesion 96. The shock waves 98 pulverize the lesion 96. The balloon wall 92 may be formed of non-compliant or compliant material to contact the intima 94.

FIG. 13 shows calcified plaque 96 pulverized by the shock waves. The intima 94 is smoothed and restored after the expanded balloon (not shown) has pulverized and reshaped the plaque into the vessel wall.

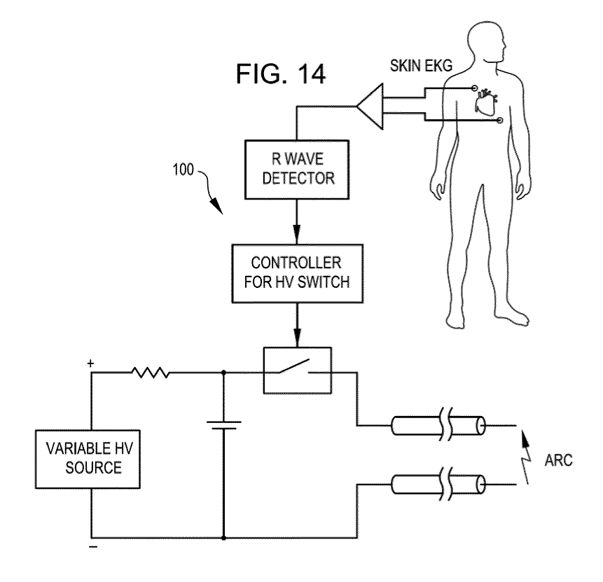

FIG. 14 is a schematic of a circuit 100 that uses the generator circuit 30 of FIG. 3 and a surface EKG 102 to synchronize the shock wave to the “R” wave for treating vessels near the heart. The circuit includes an R-wave detector 102 and a controller 104 to control the high voltage switch 32. Mechanical shock can stimulate heart muscle and could lead to an arrhythmia. While it is unlikely that shockwaves of such short duration as contemplated herein would stimulate the heart, by synchronizing the pulses (or bursts of pulses) with the R-wave, an additional degree of safety is provided when used on vessels of the heart or near the heart. While the balloon in the current drawings will provide an electrical isolation of the patient from the current, a device could be made in a non-balloon or non isolated manner using blood as the fluid. In such a device, synchronization to the R-wave would significantly improve the safety against unwanted arrhythmias.

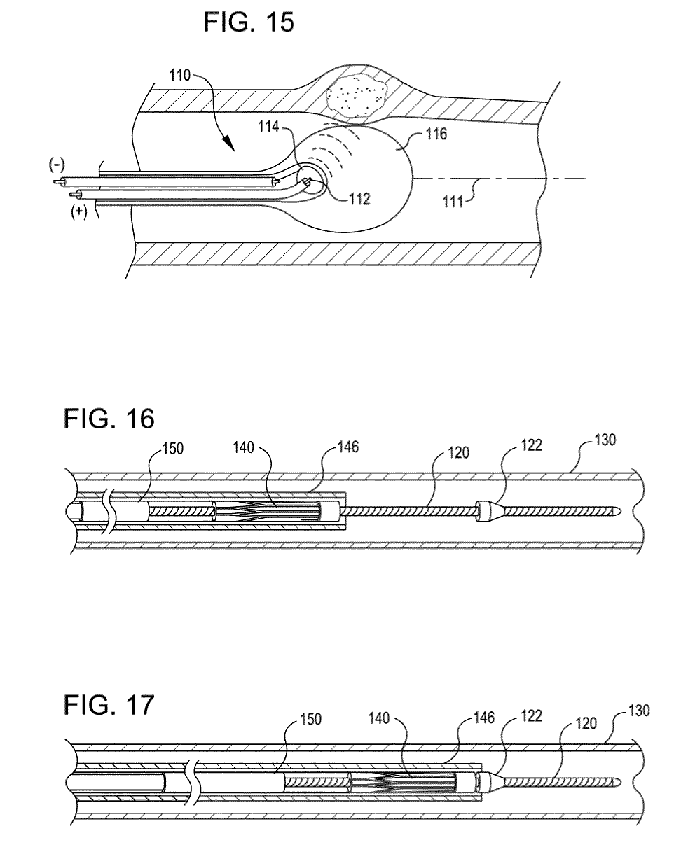

FIG. 15 shows a still further dilation catheter 110 wherein a shock wave is focused with a parabolic reflector 114 acting as one electrode inside a fluid filled compliant balloon 116. The other electrode 112 is located at the coaxial center of the reflector 114. By using the reflector as one electrode, the shock wave can be focused and therefore pointed at an angle (45 degrees, for example) off the center line 111 of the catheter artery. In this configuration, the other electrode 112 will be designed to be at the coaxial center of the reflector and designed to arc to the reflector 114 through the fluid. The catheter can be rotated if needed to break hard plaque as it rotates and delivers shockwaves.

FIG. 16 shows a guide wire 120 upon which an embolic protection basket 140 may be guided into a desired position within a vessel 130, such as an artery or vein. The guide wire 120 has a stop 122 for holding the embolic protection basket 140 in place at its desired position within the vessel 130. As an initial step in the angioplasty procedure, the embolic protection basket 140 is pushed along the guide wire 120 by a push tube 150 and advanced into position in a coronary or peripheral vessel in a manner well known in the art. The basket 140 is initially collapsed and held within an overtube 146 as it is advanced down the guide wire 120 in the collapsed state to its final desired position.

As may be noted in FIG. 17, when the embolic protection basket 140 abuts the stop 122 of the guide wire 120, it has reached its desired position. The overtube 146 may then be withdrawn while, at the same time, the push tube 150 is held in place releasing the embolic basket 140.

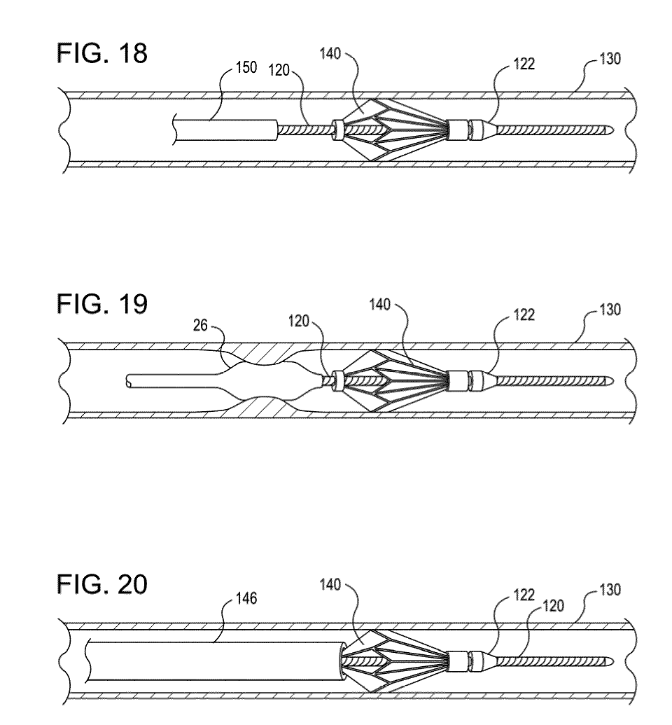

FIG. 18 shows the embolic protection basket 140 expanded and deployed in the vessel 130. The overtube has been withdrawn and the pushtube 150 is being retracted.

FIG. 19 shows the advancement of the shock wave angioplasty balloon catheter 26 over the guidewire 120 to a position of a lesion in the vessel 130 proximal to the embolic basket 140. The shock wave catheter 26 while pulverizing the calcium may dislodge calcium or embolic material resulting in debris within the vessel 130. The embolic protection basket 140 positioned downstream from the angioplasty catheter 26 will capture the debris to preclude it from clotting further down the vessel 130.

FIG. 20 shows the embolic basket 140 and contents being withdrawn into the overtube 146 after the angioplasty catheter has been removed. As indicated herein, the angioplasty catheter may be of the type previously described in the various embodiments herein. Further, embolic protection baskets of various types may be employed without departing from the present invention. Embolic protection baskets, also referred to as simply filters, which may be used herein include basket type filters with a fabric or nitinol weave, coiled-up nitinol wire, or balloons that block up or down stream of the blockage used in combination with an aspiration tube.

While particular embodiments of the present invention have been shown and described, modifications may be made, and it is therefore intended in the appended claims to cover all such changes and modifications which fall within the true spirit and scope of the invention as defined by those claims.

What is claimed is:

1. A system comprising:
a guide wire;
an embolic protection basket carried on the guide wire; and
a catheter carried on the guide wire adjacent the embolic protection basket, the catheter including an elongated carrier, a balloon about the carrier in sealed relation thereto, the balloon being arranged to receive a fluid therein that inflates the balloon, and agenerator within the balloon that forms a mechanical shock wave within the balloon.

2. The system of claim 1, wherein the generator includes at least one electrode that forms an electrical arc.

3. The system of claim 1, wherein the embolic protection basket is downstream from the catheter.

4. The system of claim 1, further including a power source that provides electrical energy to the arc generator.

5. The system of claim 1, wherein the embolic protection basket is a collapsible structure.

6. The system of claim 1, further comprising a push tube that pushes the embolic protection basket into position along the guide wire.

7. The system of claim 1, wherein the embolic protection basket is a collapsible structure and wherein the system further comprises an over tube that maintains the embolic protection basket in a collapsed condition.

8. The system of claim 1, further comprising a push tube that pushes the embolic protection basket into position along the guide wire, wherein the embolic protection basket is a collapsible structure and wherein the system further comprises an over tube that maintains the embolic protection basket in a collapsed condition as the push tube pushes the embolic protection basket into position along the guide wire.

9. The system of claim 1, wherein the guide wire includes a stop that positions the embolic protection basket on the guide wire.

10. The system of claim 1, wherein the catheter includes a lumen for being received on the guide wire.

11. A method comprising:
providing a guide wire;
inserting the guide wire into a vessel of interest of a patient;
providing an embolic protection basket;
advancing the embolic protection basket along the guide wire within the vessel;
providing a catheter including an elongated carrier, a balloon about the carrier in sealed relation thereto, the balloon being arranged to receive a fluid therein that inflates the balloon, and agenerator within the balloon that forms a mechanical shock wave within the balloon;
guiding the catheter into the vessel of the patient to a position adjacent to and up stream from the embolic protection basket;
admitting fluid into the balloon; and
causing the generator to form a series of mechanical shocks within the balloon.

12. The method of claim 1, wherein the generator includes at least one electrode that forms electrical arcs to form the mechanical shock wave, and wherein the causing step includes applying high voltage pulses to the generator.

13. The method of claim 10, wherein the embolic protection basket is a collapsible structure and wherein the step of advancing the embolic protection basket along the guide wire within the vessel is performed with the embolic protection basket maintained in a collapsed state.

14. The method of claim 11, wherein the embolic protection basket is maintained in a collapsed state by enclosing the embolic protection basket within an over tube.

15. The method of claim 12, including the further step of releasing the embolic protection basket from the collapsed state by separating the embolic protection basket from the over tube prior to applying high voltage pulses to the arc generator to form a series of mechanical shocks within the balloon.
